(19) United States
(12) Patent Application Publication
(10) Pub. No.: US 2008/0311605 A1
(43) Pub. Date: Dec. 18, 2008

(54) KIT AND METHOD TO PERFORM A BIOLOGICAL TEST DESIGNED TO EVALUATE MINERALS POTENTIAL FOR BEING BIOLEACHED

(76) Inventors: Maria de la Luz OSSES KLEIN, Santiago (CL); Paz Consuelo OSORIO DELGADO, Santiago (CL)

Correspondence Address: Maria de la Luz OSSES KLEIN;ET SEQ LLC, Suite 142, 10061 Riverside Drive, Toluca Lake, CA, 91602, US

(21) Appl. No.: 12/114,165

(22) Filed: May 2, 2008

(30) Foreign Application Priority Data
May 2, 2007 (CL) CL-1246-2007

(51) Int. Cl. C12Q 1/64; B01J 19/00; C12M 1/34

(52) U.S. Cl. 435/9; 422/61; 435/288.1

(57) ABSTRACT

A kit and method to perform a biological assay for evaluating the bioleaching potential of minerals, which allows to know the metallurgic response of minerals for geo-mining-metallurgic short and mid term planning, wherein the kit comprises a propylene tube-type container which has a line that specifies the liquid filling level, where is it possible to place a propylene sphere that floats at the liquid level and it has a plastic twist off cup on the upper extreme, while in the lower extreme or base there is a lyophilized biomass (powder) in charge of the mineral leaching.

### FIELD OF THE INVENTION

The present invention provides a Biotest for the evaluation of minerals potential for being leached by bacteria in hydro metallurgic processes of bioleaching and bio-oxidation of minerals (copper sulfides, copper oxides, gold minerals and other metals).

### BACKGROUND OF THE INVENTION

Currently, during copper minerals exploitation and processing, the geo-mining-metallurgic planning has become the tool that allows the interrelation among geological, mining and metallurgic knowledge, which permits the optimization of the process as a whole and not, as previously done, when each of these disciplines acted as an isolated link of information from one area to another and there was not a possibility of interaction, synergy or feedback among them.

This new vision has allowed the optimization of the mining planning in order to obtain the best results in the plant, for example: preparing mineral mixes, performing stock management. In turn, the metallurgic operation of plants which have this integral view, has been optimized through the particle size control of the mineral mix, processing times and all of that is done with a needed vision of the mining operation that must put into practice in the field, ore deposit, the planning developed by the geologists and metallurgists.

This is a reality in the treatment of oxidized minerals and for that purpose there are different tools to rely on, among which the predictive metallurgic tests stand out, these tests quantify the copper extraction potential by means of chemical leaching of minerals.

These tests, that are applied to daily taken samples from the ore deposit (approximately 50 samples per day), last 1 to 3 days (depending on the protocol used in each mine company), have high repeatability and are able to accomplish the objective of determining the variability of the metallurgic result for each mineral zone, whether this is polygonal (banco) or expansion, in the ore deposit depth and also in its length.

This optimum way of performing the geo-mining-metallurgic planning for the oxides hydrometallurgy, has not been yet developed to an equivalent level for the hydro metallurgic process of sulfide minerals. This is due to the lack of predictive metallurgic tests which could quantify the bioleaching extraction potential of a mineral, test that also should have the characteristics of being fast, highly repeatable and be able to represent the variability of the mineral resource.

Bioleaching can be described as the leaching of a determined raw material (mineral) by the direct or indirect action of microorganisms. In principle, the direct method can be understood as the one mediated by the action of bacteria and where the chemical reactions are catalyzed by enzymes, this option also assumes the physical contact between the microorganisms and the mineral. On the other side, the indirect mechanism is understood as the one where the chemical reactions, enzymatic and not enzymatic, occur without physical contact between the microorganisms and the mineral, even though the microorganisms play a central role in the formation of reactants that can take part in the process.

### BRIEF DESCRIPTION OF THE INVENTION

The present invention provides a Biotest as a kit, to be applied to minerals, which is a 100% biological, it is direct and reflects the direct and indirect mechanisms of bacterial leaching. It is a fast, highly repeatable, low cost and semi-quantitative biological test. Its objective is to help to determine the metallurgic response of the minerals for the geo-mining-metallurgic short term planning; therefore, the grade of the mineral can be determined and therefore which process of extraction is the most adequate (chemical leaching, bioleaching, pyro-metallurgy, etc.)

### BRIEF FIGURES DESCRIPTION

In FIG. 1, the invention can be appreciated, which consists of a tube made of a resistant and transparent material that contains a lyophilized bacterial mass in its interior, at the bottom.

FIG. 2 shows an example of the bacterial activity (or biological activity) record strip, which quantifies the metal of interest dissolution and in this manner, it is obtained the range for mineral extraction mediated by microorganisms.

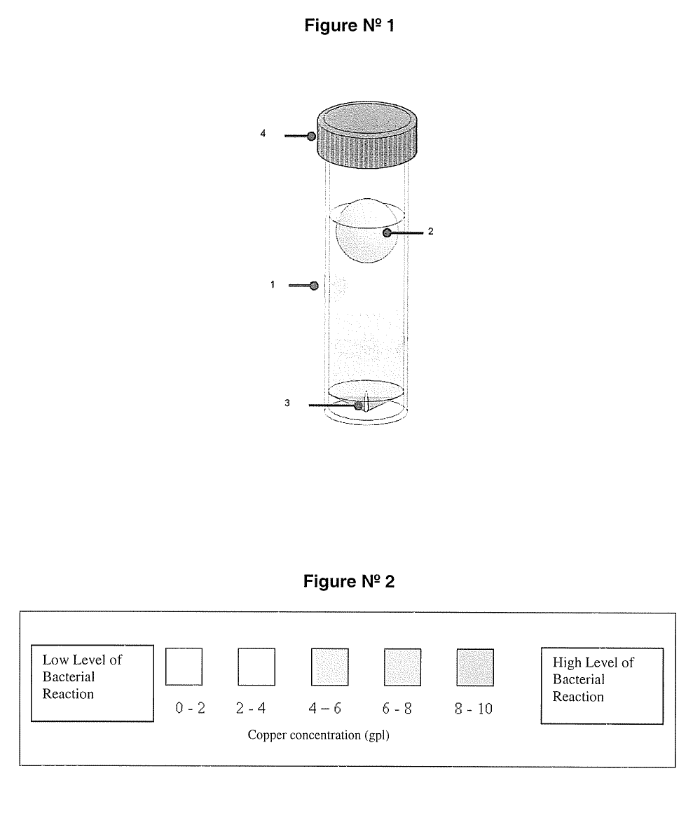

### DETAILED DESCRIPTION OF THE INVENTION

The biological test for evaluating the bioleaching potential of minerals permits to know the metallurgic response of the sulfide minerals for short term geo-mining-metallurgic planning, and also it can be used in field exploration and initial characterization of ore deposits. It is a fast assay, whose estimated answer time is from 3 to 4 days. It is low cost; it does not need additional physical-chemical or biological controls to those already performed as routine in the mining job. It has low requirement of human resources. It is a 100% biological assay, it is direct and it reflects the direct and indirect leaching mechanisms. The Biotest allows determining the variability of the metallurgic response of minerals according to their different mineralogy, grades, gangue and impurities that are present.

The present invention provides a Biotest as a kit, which consists of a propylene tube-type container (1), preferably of 10 cm height and 3 cm diameter, which has a line that specifies the liquid filling level and it has a plastic twist off cup (4) on the upper extreme.

Eventually, it can be operated with a propylene sphere (2) that floats on the liquid at the filling level inside the tube (1), whose objective is to reduce gas exchange between the exterior and interior, generating anaerobic conditions (lack of oxygen) inside the tube (4), this is accomplished due to the diameter of such sphere (2) that is slightly smaller than the inner diameter of the tube (1) allowing it to fit tight in it.

In case that aerobic conditions are required (presence of oxygen), the only necessary condition to achieve it, is just to remove the sphere (2) from the tube (1) and operate in the same manner, but without using it.

At the lower extreme of the tube (1) or base, it is placed a determined mass of biomass (3) from the lyophilized bacterial composition (powder), this biomass consists of typical bacterial consortium found in minerals, they are responsible of the bioleaching process, and when in contact with the mineral sample and the leaching solution (or acid water at pH 1.4-2.0) will catalyze the metal extraction if there are minerals with the capability of being bioleached.

The amount of mineral to be used must not be over 15 g nor lower than 5 g. The solution volume must not be over 30 ml nor lower than 19 ml.

Eventually, the test could include a compound (e.g. enzymes or nutrients) that accelerates or improves the microorganisms' metabolism.

### Method of Use:

a) The use of this product must be performed in a laboratory (work place), it can eventually be used in the field if minimal working conditions (sterile conditions, surgical gloves, good sample handling) are met.
The method of use consists of:
   Open the tube (1);
   Remove the sphere (2); in case that the test contains one;
   Add the sample mineral and the leaching solution (or acid water) in the tube (1) up to the specified level and keep it in a vertical position;
   Place the sphere (2) in the tube only if anaerobic conditions are required;
   Close the tube (1);
   Keep the tube (1) in vertical position, making sure that there is not exogenous contamination and incubate it in such position at room temperature during at least 3 days.

b) The results will be of semi-quantitative nature, it will deliver the metallurgic extraction value from 0 to 100% in ranges, which will depend on the type of mineral being tested.

c) The result is obtained by comparing the color of the solution that has already reacted in the tube (1) with the colors printed on the strip (5) attached to the lower half of the outer surface of the tube (1). The strip indicates the range of metallurgic dissolution of the sample by means of different color tones. For example, the strip colors for copper vary from light to dark blue, where the lighter color means lower copper concentration in solution, while the darker color means higher metal dissolution and therefore higher copper concentration in solution. By relating this result of concentration in solution of the metal of interest, with the initial metal content in the mineral tested, mineral grade, (data obtained during routine control in mine work) and also with the volume of the leaching solution added in the Biotest, the potential dissolution of copper is obtained.

### EXAMPLE

It is provided a Biotest without sphere to carry out an aerobic process. The Biotest is opened and a mineral sample, that has a minimal mass of 5 g and a maximum mass of 15 g-10# (particle size less than 10 mesh sieve) is placed in the tube, then a volume of 10 ml to 30 ml of leaching solution is added in the Biotest tube, which must be kept in vertical position, and subsequently the cap is placed on the tube to close the Biotest. Afterwards, the Biotest must be kept in vertical position, making sure that there is not exogenous contamination, and incubate it in that position at room temperature dulling at least 3 days. The tube, always in vertical position, must be agitated at least twice a day.

In case that the process requires anaerobic conditions, the same steps of mineral mass and leaching solution volume must be followed, but a propylene sphere must be placed in the tube before closing it, to ensure lack of oxygen.

1. A kit to perform a biological assay for evaluating the bioleaching potential of minerals, which allows to know the metallurgic response of minerals for geo-mining-metallurgy short and mid term planning, wherein the kit comprises a propylene tube-type container which has a line that specifies the liquid filling level, where is it possible to place a propylene sphere that floats on the liquid at the filling level and it has a plastic twist off cup on the upper extreme, while in the lower extreme or base there is a lyophilized biomass (powder) in charge of the mineral leaching.

2. The kit to perform a biological assay for evaluating the bioleaching potential of minerals according to claim 1, wherein such biomass comprises a bacterial consortium responsible of the bioleaching process, which catalyzes the bio-extraction process by being in contact with the mineral sample, if the sample has minerals with the potential of being bioleached.

3. The kit to perform a biological assay for evaluating the bioleaching potential of minerals according to claim 1, wherein the propylene sphere is used to reduce the gaseous exchange between the exterior and interior of the tube, to generate anaerobic conditions inside the tube (or aerobic conditions when it is not used), because it fits tight inside the tube and furthermore, the base of the tube has conical shape to generate an extreme anaerobic condition (lack of oxygen).

4. The kit to perform a biological assay for evaluating the bioleaching potential of minerals according to claim 1, wherein the test is fast, whose estimated response time is 3 to 4 days; it is low cost, it does not need physical-chemical controls nor microbiological controls; it is a 100% biological test, it is direct and reflects the direct and indirect mechanisms of bacterial leaching.

5. The kit to perform a biological assay for evaluating the bioleaching potential of minerals according to claim 1, wherein such minerals comprise copper sulfide, gold, molybdenum, zinc, and others.

6. The kit to perform a biological assay for evaluating the bioleaching potential of minerals according to claim 5, wherein the amount of sulfide minerals to be assayed has a minimal mass of 5 g and a maximum mass of 15 g-10# (particle size less than 10 mesh sieve), and the volume of the leaching solution must be from 10 ml to 30 ml.

7. A method to perform a biological assay for evaluating the bioleaching potential of minerals, which permits to know the metallurgic response of minerals for geo-mining-metallurgy short and mid term planning, and which comprises the following steps:
i) open the propylene tube-type container which has a line that specifies the liquid filling level;
ii) Remove the propylene sphere (in case that anaerobic conditions are needed) whose objective is to reduce the gaseous exchange between the exterior and exterior of the tube, generating anaerobic conditions, because the sphere fits tight in the inner diameter of the tube;
iii) add the sample mineral and the leaching solution (or acid water);
iv) place the propylene sphere in the tube, close it while keeping it vertically, making sure there are sterile conditions, and incubate it at room temperature.
v) the result is obtained by comparing the color of the solution that has already reacted in the tube with the colors printed on the strip attached to the outer surface of the tube, which indicates the range of metallurgic dissolution of the sample by means of different color tones established for each mineral.

8. The method to perform a biological assay for evaluating the bioleaching potential of minerals, according to claim 7, wherein the step of adding the sample mineral and the leaching solution (or acid water), consists of adding a minimal mass of 5 g and a maximum mass of 15 g-10# (particle size less than 10 mesh sieve), and the volume of the leaching solution must be from 10 ml to 30 ml.

9. The method to perform a biological assay for evaluating the bioleaching potential of minerals, according to claim 7, wherein the results are of semi-quantitative nature, they consist of the metallurgic extraction value from 0 to 100% in ranges that depend on the type of mineral being tested.
